(19) United States
(12) Patent Application Publication
(10) Pub. No.: US 2023/0012749 A1
(43) Pub. Date: Jan. 19, 2023

(54) CHEST WOUND SYSTEM

(71) Applicant: Moldoveanu Designs LLC, Austin, TX (US)

(72) Inventor: Andrei Gavrilut, Bristol (GB)

(21) Appl. No.: 17/859,704

(22) Filed: Jul. 7, 2022

Related U.S. Application Data
(60) Provisional application No. 63/221,055, filed on Jul. 13, 2021.

(51) Int. Cl. A61M 1/04; A61M 39/24

(52) U.S. Cl. CPC A61M 1/04; A61M 39/24; A61M 2039/2446; A61M 2039/2406; A61M 2210/101; A61M 2039/0229

(57) ABSTRACT

A system for treatment of an open wound in a chest of a patient is disclosed. The system includes a seal membrane configured to adhere to the chest and having an air passage. The system also includes a mechanism removably coupled to the seal membrane over the air passage. The mechanism is configured to allow air to pass through the mechanism in a first direction and not to pass in a second direction opposite the first direction.

### CROSS-REFERENCE TO RELATED APPLICATIONS

This application claims the benefit of U.S. application 63/221,055 filed on Jul. 13, 2021, which is hereby incorporated herein in its entirety.

### STATEMENT REGARDING FEDERALLY SPONSORED RESEARCH OR DEVELOPMENT

Not applicable.

### BACKGROUND

### Field

The present invention generally relates to wound dressings, in particular a dressing for chest wounds.

### Description of the Related Art

Injuries to the chest can have effects on multiple organs, such as the lungs and heart, as well as on the structure and function of the chest cavity. One condition of particular importance is “pneumothorax,” also referred to as a “collapsed lung,” wherein air collects in the pleural space between the lung and chest wall. As expansion of the rib cage no longer expands the lungs, pneumothorax leads to a steadily worsening oxygen shortage and low blood pressure and can be fatal.

A pneumothorax may be classified as “open” or “closed.” In an open pneumothorax, there is a passage from the external environment into the pleural space through the chest wall. A closed pneumothorax is when the chest wall remains intact. Tension pneumothorax is a rapidly-deteriorating critical condition that usually develops following a traumatic open pneumothorax. When air builds up in the pleural space, it creates a positive pressure within the chest cavity. As a result, mediastinal structures that contain the heart and major blood vessels are displaced and compressed against the opposite lung. This in turn results in compromised cardiopulmonary function, inadequate tissue oxygenation (shock), and death. Tension-pneumothorax can be fatal within minutes of the injury.

Conventional field treatment for a closed pneumothorax is usually achieved insertion of a 14 G needle in the affected side of the chest in order to relieve air pressure. To prevent a tension pneumothorax, a functional vented chest seal is immediately applied over the needle. In the case of an open chest wound, a functional vented chest seal is applied directly over the open pneumothorax. This type of dressing adheres to the chest wall and allows air to escape but not to enter the chest. Chest injuries frequently involve internal bleeding and the chest seal becomes clogged with blood and ceases to function. This necessitates “burping” the seal to allow air to escape directly, which reduces the adhesion of the seal to the chest and degrades its performance and benefit. Furthermore, the burping is only a momentary relief from the accumulation of air in the pleural cavity and must be repeated periodically, with an increasing degradation of the seal's effectiveness.

### SUMMARY

It is desirable to provide a wound dressing that incorporates a mechanism that allows air to escape but not enter the pleural cavity and is easily cleaned and restored to proper operation.

In certain embodiments, it is desirable to provide a system for treatment of an open wound in a chest of a patient. The system includes a seal membrane configured to adhere to the chest and having an air passage. The system also includes a mechanism removably coupled to the seal membrane over the air passage. The mechanism is configured to allow air to pass through the mechanism in a first direction and not to pass in a second direction opposite the first direction.

In certain embodiments, it is desirable to provide a method for restoring a chest wound system to proper operation. The method includes the step of removing a first mechanism, which is configured to allow air to pass through the mechanism in a first direction and not to pass in a second direction opposite the first direction, from a fitting fixedly coupled to a seal membrane. The method also includes the step of installing a second mechanism, which is interchangeable with the first mechanism, on the fitting.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a conventional chest seal.

FIG. 2 depicts an exemplary chest wound system, according to certain aspects of the present disclosure.

FIG. 3 depicts an exemplary fitting coupled to a seal, according to certain aspects of the present disclosure.

FIG. 4 depicts an exemplary mechanism for controlling the flow of air through the system, according to certain aspects of the present disclosure.

FIG. 5 is a cutaway view of the vent assembly, according to certain aspects of the present disclosure.

### DETAILED DESCRIPTION

The following description discloses embodiments of a disinfection apparatus.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form to avoid obscuring the concepts of the subject technology. Like, or substantially similar, components are labeled with identical element numbers for ease of understanding.

There is a need for an improved chest seal, especially for the treatment of battle wounds that involve significant bleeding from a penetration wound. Blood often enters the air release feature of conventional chest seals and coagulates, thereby clogging the air passage and interfering with the release of air. Providing the ability to remove and clean, or replace, the air release mechanism is a significant improvement in the treatment of open chest wounds prior to arrival at a hospital or other medical facility.

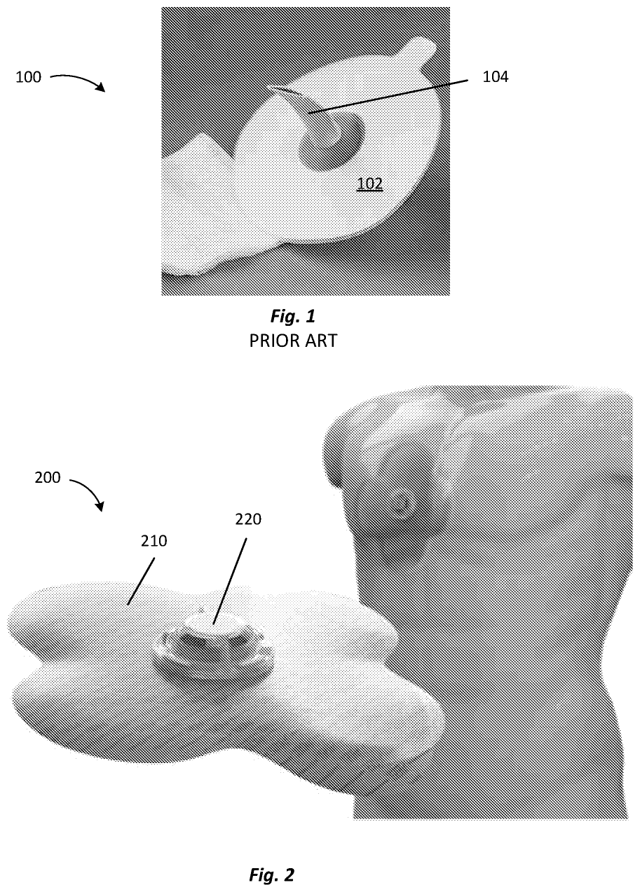

FIG. 1 depicts a conventional chest seal 100. The seal 102 is coated with an adhesive on the underside (in this view) so as to be adhered to the chest of an injured person. The valve 104 is a flexible tube with a natural position wherein the tube is collapsed so as prevent the passage of air from the environment into the pleural cavity of the patient. Air can pass outward from the pleural cavity to the environment through the tube. Blood from the pleural cavity will enter the tube and clog the tube. It is not possible to clean the valve 104 after the chest seal 100 is applied to a patient.

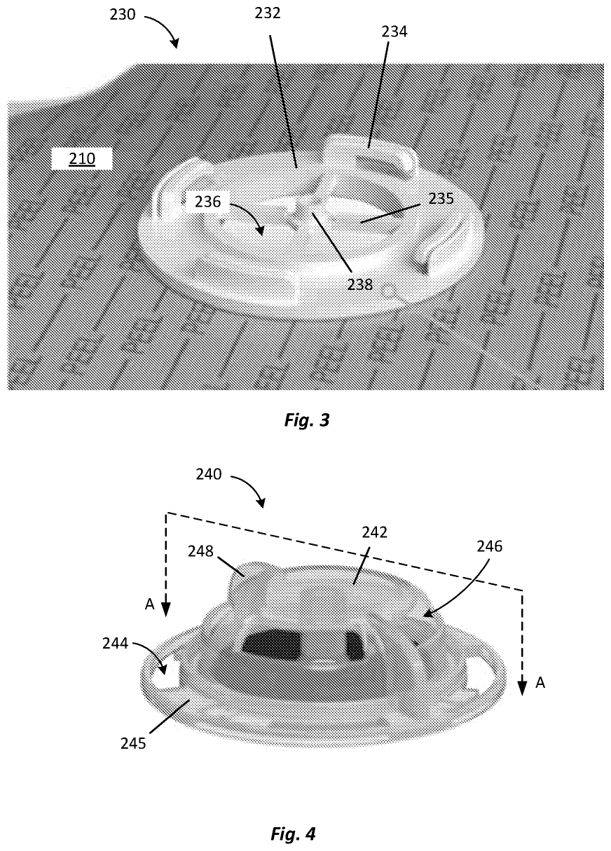

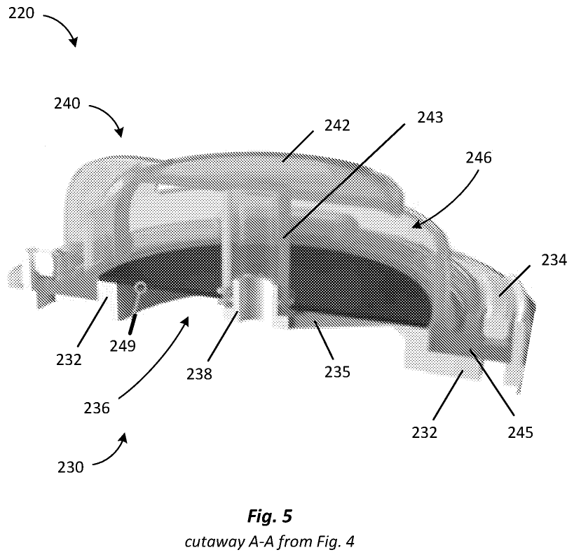

FIG. 2 depicts an exemplary chest wound system 200, according to certain aspects of the present disclosure. The system 200 comprises a seal membrane 210 that is configured to adhere to the chest of a patient. The seal membrane 210 comprises an air passage (not visible in FIG. 2). The system 200 further comprises a vent assembly 220 selectably coupled to the seal membrane 210, wherein a portion of the vent assembly can be removed from and re-attached to the membrane seal 210. The vent assembly 220 is disposed over the air passage 216 and configured to allow air to pass through the mechanism in a first direction from the pleural cavity through the air passage to the environment and not to pass in a second direction opposite the first direction. The vent assembly 220 comprises a fitting 230 and a mechanism 240 that are discussed in further detail with respect to FIGS. 3-4.

In certain embodiments, the seal membrane 210 comprises a plurality of lobes to provide an enhanced ability to conform to the chest surface. FIG. 2 depicts an exemplary seal membrane 210 with four lobes. In certain embodiments, the seal membrane 210 has 2 lobes. In certain embodiments, the seal membrane 210 has 3 lobes. In certain embodiments, the seal membrane 210 has more than 4 lobes.

In certain embodiments, the vent assembly 220 is disposed on a top surface of the seal membrane 210, as shown in FIG. 2, and a layer of adhesive (not visible in FIG. 2) is disposed on a bottom surface of the seal membrane 210 that is opposite to the top surface. In certain embodiments, a removable cover sheet (not shown in FIG. 2) is provided over the layer of adhesive to facilitate storage, the cover sheet being removed prior to adhering the system 200 to the patient.

FIG. 3 depicts an exemplary fitting 230 coupled to the seal membrane 210, according to certain aspects of the present disclosure. In certain embodiments, the fitting 230 is non-detachably coupled to the seal membrane 210. The fitting 230 comprises a body 232 and an internal opening 236 that, when the fitting 230 is attached to the seal membrane 210 over the opening in the seal membrane 210, air can pass through the fitting 230. The fitting 230 further comprises a center pin 238 and one or more spokes 235 and an attachment feature, e.g., the prongs 234, that is configured to detachably engage the mechanism 240.

FIG. 4 depicts an exemplary mechanism 240 for controlling the flow of air through the vent assembly 220, according to certain aspects of the present disclosure. The mechanism 240 comprises a housing 242 having vent openings 246 through the housing 242 and a mating feature, e.g., the openings 244 in flange 245, that is configured to engage the attachment feature, e.g., the prongs 234, of the fitting 230. In certain embodiments, the mating feature and the attachment feature are configured such that the mechanism 240 can be selectively removed and re-installed on the fitting 230. In certain embodiments, the mechanism 240 is configured to be manually, i.e., without tools, attached to and removed from the fitting 230. In certain embodiments, the mechanism 240 is configured to be removed from and the same mechanism 240 re-attached to the fitting 230 without a tool or use of additional material, e.g., adhesive or sealant. In certain embodiments, the housing 242 comprises one or more tabs 248 that facilitate application of torque to attach or detach the mechanism 240 from the fitting 230.

FIG. 5 is a cutaway view of vent assembly 220, with the mechanism 240 attached to fitting 230, along the section A-A of FIG. 4, according to certain aspects of the present disclosure. The flange 245 of the mechanism 240 forms an essentially airtight seal with the fitting 232. The mechanism 240 comprises an internal post 226 that projects from the interior of housing 242 and, in certain embodiments, mates with the center pin 238. In the example embodiment, a flexible disk 249 is fixedly coupled to post 243 while the periphery of the disk 249 is free to move up and down, i.e., not attached to the housing 242. In the absence of external force, disk 249 is in a first position with the perimeter of the disk 249 in contact with the fitting 232 around the opening 236. When gas pressure on the bottom side of the disk is greater than the gas pressure on a top side of the disk 249, which is opposite the bottom side, disk 249 will flex upward at the perimeter to a second position thereby forming a gap between the disk 249 and the fixture 212 such that air can flow outward from the pleural cavity of the patient by passing between the opening 236 of fitting 232 and past the displaced outer edge of the disk 249 between the fitting 232 and the disk 249, and out through opening 246. If the air pressure within the pleural cavity is equal to or lower than the ambient air pressure, the perimeter of disk 249 will be returned to the first position with sealing contact with fitting 232 that prevents inward air flow. In certain embodiments, the one or more spokes 235 provide support for the disk 249 in the first position, thereby preventing deformation that would disturb the seal between the disk 249 and fitting 232.

In certain embodiments, the housing 242 is configured such that the disk 249 seals in the first position against a portion of the housing 242 and the housing 242 forms a generally airtight seal to the fitting 232. Alternate constructions that provide an equivalent function of flow control, permitting air to pas in the outward direction while inhibiting flow in the inward direction will be apparent to those of skill in the art.

The mechanism 240 is configured to be cleanable such that foreign matter, e.g., blood or dirt, can removed from an interior of the mechanism 240 so as to restore passage of air in the first outward direction and prevent passage of air in the second inward direction. In certain embodiments, the mechanism 240 is configured to be cleanable by separating the mechanism 240 from the fitting 230 and flushing the mechanism 240 with water or other liquid, e.g., isotonic crystalloid solution, chlorhexidine solution, sterile water, bottled drinking water, or tap water. The interior of housing 242 is accessible by pushing disk 249 aside, thereby providing easy and complete access to the sealing perimeter of disk 249, which is the area most likely to be clogged or contaminated.

In certain embodiments, the mechanism 240 is configured to be fully inspectable, e.g., all interior spaces and surfaces can be viewed, with non-destructive movement of components such as the flexible disk 249, while removed from the fitting to verify that foreign material has been sufficiently removed and the mechanism 240 is functional before being re-installed.

It is also possible, in certain embodiments, to replace a clogged mechanism 240 with an interchangeable new or previously cleaned mechanism 240. In some instances, the care provider may not have the time to remove and clean a clogged mechanism 240, so a swap with a brand-new mechanism 240 is a quicker and simpler fix. In certain embodiments, one or more spare interchanageable mechanisms 240 are provided with the chest wound system 200.

In summary, the removable and interchangeable mechanism 240 allows a car provider to resolve issues with proper operation of the chest wound system 200 without compromising the adhesive on an otherwise-working system 200. The ability to quick repair or replace the mechanism 240 reduces the time required to correct a problem, benefiting both the patient and the operator, and reduces the likelihood of needing a more invasive procedure, e.g., such as needle-chest-decompression, due to less build-up of air inside the pleural space.

### Aspects

<List Original Claims for Retention, After Claims are Final>

Headings and subheadings, if any, are used for convenience only and do not limit the invention.

Reference to an element in the singular is not intended to mean “one and only one” unless specifically so stated, but rather “one or more.” Use of the articles “a” and “an” is to be interpreted as equivalent to the phrase “at least one.” Unless specifically stated otherwise, the terms “a set” and “some” refer to one or more.

Terms such as “top,” “bottom,” “upper,” “lower,” “left,” “right,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Although the relationships among various components are described herein and/or are illustrated as being orthogonal or perpendicular, those components can be arranged in other configurations in some embodiments. For example, the angles formed between the referenced components can be greater or less than 90 degrees in some embodiments.

Although various components are illustrated as being flat and/or straight, those components can have other configurations, such as curved or tapered for example, in some embodiments.

Pronouns in the masculine (e.g., his) include the feminine and neuter gender (e.g., her and its) and vice versa. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “operation for.”

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such as an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

Although embodiments of the present disclosure have been described and illustrated in detail, it is to be clearly understood that the same is by way of illustration and example only and is not to be taken by way of limitation, the scope of the present invention being limited only by the terms of the appended claims.

What is claimed is:

1. A system for treatment of an open wound in a chest of a patient, the system comprising:
a seal membrane configured to adhere to the chest and comprising an air passage; and
a mechanism removably coupled to the seal membrane over the air passage, the mechanism configured to allow air to pass through the mechanism in a first direction and not to pass in a second direction opposite the first direction.

2. The system of claim 1, wherein the mechanism comprises a plurality of interchangeable mechanisms.

3. The system of claim 1, further comprising a fitting fixedly coupled to the seal membrane and comprising an attachment feature, wherein the mechanism comprises a mating feature configured to engage the attachment feature so as to retain the mechanism on the fitting.

4. The system of claim 3, wherein mating feature and the attachment feature are configured such that the mechanism can be selectively removed from and re-installed on the fitting.

5. The system of claim 4, wherein the mechanism that is removed from the fitting can be re-installed on the fitting without a tool or additional material.

6. The system of claim 4, wherein the mechanism is configured to be cleanable so as to restore passage of air in the first direction and prevent passage of air in the second direction.

7. The system of claim 6, wherein the mechanism is configured to be fully inspectable while removed from the fitting.

8. The system of claim 3, wherein the mechanism comprises a flexible disk having a first position that prevents air from passing in the second direction and a second position that allows air to pass through the mechanism in the first direction.

9. The system of claim 8, wherein the flexible disk is configured to move from the first position to the second position when a first gas pressure on a bottom side of the disk is greater than a second gas pressure on a top side of the disk and to move from the second position to the first position when the first gas pressure is equal to or lower than the second gas pressure.

10. The system of claim 8, wherein:
the disk forms a generally airtight seal against the fitting in the first position; and
the fitting comprises a support arm configured to support the flexible disk in the first position.

11. A method for restoring a chest wound system to proper operation, comprising the steps of:
removing a first mechanism, which is configured to allow air to pass through the mechanism in a first direction and not to pass in a second direction opposite the first direction, from a fitting fixedly coupled to a seal membrane; and
installing a second mechanism, which is interchangeable with the first mechanism, on the fitting.

12. The method of claim 11, further comprising the step of cleaning the removed first mechanism to remove foreign material that is clogging the removed first mechanism, wherein the second mechanism is the cleaned first mechanism.

13. The method of claim 12, further comprising the step of fully inspecting the cleaned first mechanism before re-installing the cleaned first mechanism.

14. The method of claim 12, wherein:
all steps are executed while the seal membrane remains adhesively attached to a chest of a patient; and
the foreign material comprises blood of the patient.

15. The method of claim 11, wherein the steps of removing the first mechanism is performed without the use of a tool.

16. The method of claim 11, wherein the steps of installing the second mechanism is performed without the use of a tool or additional material.
